(19) United States
(12) Patent Application Publication
(10) Pub. No.: US 2008/0050775 A1
(43) Pub. Date: Feb. 28, 2008

(54) Methods for Recovering Products From Plants

(76) Inventors: John N. Burr III, Rancho Santa Fe, CA (US); Carl Miller, Raleigh, NC (US)

Correspondence Address: BioTechnology Law Group;c/o PortfioloIP, P.O.Box 52050, Minneapolis, MN, 55402, US

(21) Appl. No.: 11/578,285

(22) PCT Filed: Apr. 11, 2005
(86) PCT No.: PCT/US05/12117

§ 371 Date: Aug. 9, 2007

Related U.S. Application Data
(60) Provisional application No. 60/562,072, filed on Apr. 13, 2004.

(51) Int. Cl. A01H 5/00; C12N 15/00; C12N 5/04

(52) U.S. Cl. 435/69.1; 435/410; 800/298

(57) ABSTRACT

Described are methods and compositions for producing and isolating desired products (for example, one or more protein, peptide, or pharmaceutical compound species) in transgenic plants engineered to produce the desired product(s). The desired products are isolated from the transgenic plants by contacting plant tissue with an enzyme preparation under conditions effective to hydrolyze the cell wall of the cells comprising the plant tissue. In preferred embodiments, the one or more enzyme species used to effect cell lysis are expressed in the transgenic plant.

### BACKGROUND OF THE INVENTION

1. Field of the Invention

The invention relates generally to the production and recovery of plant-made products, and more specifically to recovery of products made in transgenic plants.

2. Background Information

Pharmaceutical molecules, and other important amino acids and polypeptides are typically produced either by fermentation processes or by chemical synthesis. The ability to insert a foreign gene or one or more members of a multiple gene pathway encoding for the production these molecules into agricultural crops (plants) will allow for the production of such molecules directly in the transgenic plants. The resulting plant-made products, or “PMP's”, which include pharmaceutical products such as small molecule drugs, protein, and peptides, can have a substantial manufacturing cost advantage vis a vis conventional manufacturing methods. Regardless of the type of production process, be it fermentation, chemical synthesis, or production on a transgenic crop, the resulting desired products must be isolated, and then preferably purified to substantial homogeneity (at least in the context of pharmaceutically active molecules) from the production milieu. The isolation and purification phase has been one of the prime limitations in the commercialization of PMP's. Conventional processing of crop materials separates the crop into discrete fractions, one of which typically contains the PMP. Even with this gross fractionation, it is still extremely difficult, tedious, and expensive to isolate the target pharmaceutical molecule from the remaining plant materials.

In the context of pharmaceuticals, proteins, and peptides, such as therapeutic antibodies, proteins such as human growth hormone, insulin, erythropoietin, granulocyte colony stimulating factor, and small molecule drugs (e.g., six carbon and five carbon sugar based molecules and their polymers), and in various combinations with amino acids or as precursor molecules for molecular synthesis into the final product, as well as industrial proteins and polypeptides such as antibodies and enzymes for diagnostic, analytic, and industrial applications, can be produced or carried out in transgenic agricultural crops. This offers the potential of making processing feasible as well as reducing manufacturing costs, thereby providing a viable alternative to conventional processes, for example, fermentation-based processes. The lower costs can translate to an increased availability of the bio-product to a broader population. The manufacturing costs for a fermentation-based process are roughly split 50:50 between production phase and the recovery/purification process. The production of the desired target molecule in a transgenic crop dramatically lowers the cost of the production phase but is often associated with a higher recovery/purification cost than encountered by fermentation-based processes. The higher cost is predominantly due to a lower initial concentration of the target bio-product in the production milieu and the characteristics of the diluent plant material. The concentration of a bio-product in a fermentation process is generally in the range of 2-40 grams/liter of fermentation broth, or 1-15% on a solids basis. The concentration of target bio-molecule in a transgenic crop is typically a factor of 10-1000 times more dilute on a solids basis; 0.001-0.1%.

Typically, recovery of a bio-product from fermentation broth involves an initial cell disruption step if the molecule is not secreted, followed by cell separation, intermediate flocculation or bulk purification and concentration by ultrafiltration. A final purification by chromatography or repeated recrystallizations is typically required for a therapeutic or industrially important PMP molecule species. The transgenic crop produced bio-molecule is always produced intracellularly. Recovery of the desired PMP can be from capturing the exudates and/or via the crop requiring a cell disruption step prior to extraction. Plant cell walls are thicker and have greater structural integrity than microbial cell walls and require more force in the disruption process. The rigorous conditions—energy input, heat, sheer, and high or low pH required to break plant cell walls, oftentimes results in degradation of the target bio-molecule, which is associated with yield losses and sometimes makes downstream purification processes more difficult. Additionally, the bulky nature of the plant material may require a comminution step prior to cell disruption, and large volumes of water are needed both to make slurry for disruption and to ensure adequate extraction of the desired bio-molecule. The large equipment size, energy required, and physical and chemical losses result in a far more expensive recovery process for the PMP and far outweigh the cost advantage in the initial production phase. The net result is the potential low cost production and broader availability for the target bio-molecule is not realized.

### SUMMARY OF THE INVENTION

The present invention relates to the selective use of enzymes to hydrolyze plant cell wall material along with other contaminating plant materials to greatly facilitate the isolation of a target, or desired, molecular species. The hydrolytic enzymes may be derived from various sources, e.g., bacteria, fungi, etc., and they may be transgenically produced in the plant tissue from which the PMP is isolated, or they may be produced from another source (including a recombinant source) and applied alone or in various combinations. As will be appreciated, the enzymes used include those having an amino acid sequence as occurs naturally, as well as enzymes that have been engineered to alter or enhance one or more properties of the enzyme (e.g., pH and/or temperature optimum, cofactor requirements, substrate specificity, catalytic rate, thermal and/or pH stability, etc.).

Plant cell wall hydrolyzing enzymes have been proposed to be used in certain industrial applications, including bio-pulping and bleach-boosting in pulp and paper applications, bio-scouring to remove the cotton primary cell wall, retting of flax, biomass hydrolysis to inexpensively produce sugars for fermentation processes and viscosity control in fruit juice processing. However, plant cell wall-hydrolyzing enzymes have not been proposed to be used in recovery of PMPs. The enzymatic hydrolysis of the host plant cell wall in a PMP application will: 1) obviate the need for rigorous cell wall disruption equipment; 2) allow for a greater release of the desired target molecule species; 3) result in fewer losses due to product degradation; and 4) result in a decrease in the volume of water used for processing and extraction. The result is an economically favorable recovery process to complement the low-cost associated with using a PMP production phase, resulting in a lower overall manufacturing cost and broader availability of the desired bio-molecule(s). It is envisioned that depending upon the plant organ or tissue used for production, the enzymatic cell wall disruption step of the invention could be employed as an initial processing step or following a conventional post-harvest fractionation of the plant material to remove contaminants in the isolation and purification process.

Accordingly, in one aspect the present invention provides methods to isolate a product such as a protein, peptide, or pharmaceutically active compound from a plant. In some embodiments, the plant is a transgenic plant that expresses a gene product (e.g., a messenger RNA, a peptide or polypeptide transcribed from an mRNA, etc.) from an exogenous nucleic acid molecule to produce the plant-made product. The plant-made product may result directly from the expression of the exogenous nucleic acid molecule as a result of the product being encoded thereby. Examples of such products include proteins (e.g., antibodies (or portions or fragments thereof), enzymes, protein-based hormones, cytokines, etc.) and peptides. In other embodiments, the plant-made product results indirectly from the expression of the exogenous nucleic acid molecule. By this is meant that the resulting plant-made product itself is not encoded by the exogenous nucleic acid molecule introduced into the plant using recombinant techniques, but results from the action of a protein (or series of proteins) encoded by one or more exogenous nucleic acid molecules, alone or in conjunction with the action of one or more endogenous proteins. Examples of such molecules include small molecule drugs or pro-drugs, examples of which include small molecule antibiotic drugs, small molecule chemotherapeutic agents, etc. Thus, in these embodiments, the plant-made product is produced in at least one tissue or organ of the transgenic plant. The plant, or the particular tissue(s) or organ(s), as the case may be, are then contacted with an enzyme preparation (which may contain a single enzyme species or a combination of enzyme of species) under conditions effective to hydrolyze one or more components of the cell wall of cells of the plant tissue(s). In preferred embodiments of this and other processing aspects of the invention, the enzyme(s) can be used, for example, at a concentration of between 0.1% and 10% weight/weight. The plant-made product is then collected, and thus isolated. If desired, one or more subsequent purification steps adapted to purify the particular plant-made product can be employed, preferably to purify the plant-made product to substantial homogeneity, i.e., to “substantially purify” the plant-made product.

In certain preferred embodiments, such transgenic plants further include another exogenous nucleic acid molecule that encodes an enzyme for hydrolyzing one or more cell wall components. As with the exogenous nucleic acid molecule that results in the direct or indirect production of the desired plant-made product, the enzyme-encoding gene may, if desired, be constitutively or inducibly expressed, or expressed in a particular developmental or tissue-specific context by operative linkage with a suitable control or regulatory element, or combination of control elements, as those in the art will appreciate.

A related aspect concerns transgenic plants that include at least one exogenous nucleic acid molecule that encodes an enzyme capable of degrading a plant cell wall component, particularly when plant material containing the expressed enzyme are placed under conditions that promote the hydrolytic activity of the enzyme. Such plants may further include a second exogenous nucleic acid molecule, the expression of which results in production of a plant-made product (e.g., an exogenous protein, a peptide, a small molecule drug or pro-drug, etc.) in at least one tissue of the transgenic plant.

Another aspect of the invention relates to methods for enhancing recovery of a plant-made product, be it one that results directly or indirectly from the expression of an exogenous nucleic acid molecule or one that is endogenous to the particular plant. In this aspect, the plant producing the plant-made product in also a transgenic plant. Here, the plant contains at least one tissue that expresses an exogenous nucleic acid molecule that encodes an enzyme that, under conditions effective to hydrolyze plant cell walls, hydrolyses cell walls of at least a portion of the cells of tissue(s) in which the enzyme is expressed. After harvesting, the transgenic plant, or selected portion(s) thereof (i.e., those expressing the enzyme encoded by the particular transgene construct), are placed under conditions effective to hydrolyze plant cell walls, at least in part. In other words, the plants (or portion(s) thereof) are placed under conditions that allow the enzyme to hydrolyze tat least some portion of the plant cell walls of the tissue, thereby enhancing recovery of the plant-made product contained in the tissue being treated. If desired, the plant-made product may then be further purified. In some preferred embodiments, the plant-made product is produced in the transgenic plant as a result of expression of an exogenous gene. Such production may result directly from expression of the transgene, or as a result of the action of the gene product(s) encoded by the transgene(s) responsible for production of the particular plant-made product.

### DETAILED DESCRIPTION OF THE INVENTION

The present invention is based on the discovery that certain enzymes can be used to recover PMPs from plants, particularly transgenic plants engineered to produce a desired product (e.g., a protein or peptide), be it one encoded by an exogenous gene or one that results from the action of a gene product (e.g., a protein, RNA, etc.) encoded by an exogenous nucleic acid molecule. Cellulases, hemicellulases, pectinases, lignases, and amylases, either separately or in combination, are known to effect a near total hydrolysis of plant cellular material. The selective and mild nature of such enzymes results in hydrolysis of the plant cell wall with little or no physical or chemical degradation of non-cell wall material. Provided herein are enzyme-facilitated processes for the extraction of a desired PMP species, for example, a protein (e.g., an enzyme, cytokine, antibody chain, hormone, etc.), peptide, or small molecule drug, from a transgenic plant. The transgenic plant can be a transgenic crop plant, for example, a transgenic canola, corn, soy, tobacco, arabidopsis, cotton, duckweed, wheat, alfalfa plant, a transgenic grass plant, a transgenic vegetable plant, or a transgenic tree. Indeed, in accordance with this specification, any dicot or monocot suited for production of the particular PMP can be engineered to express the desired transgene(s) using art-known techniques.

Cell wall hydrolysis can be carried out, for example, at a temperature ranging from 20 C-90 C and a pH from 3-9. The hydrolytic enzyme is typically used at a concentration of between 0.1% and 10% (weight/weight), for example 0.5%-2%. It will be understand that factors such as temperature, holding time, reaction vessel holding capacity, dose, and pH, can be varied to find optimal conditions for extracting the PMP.

Accordingly, in one embodiment, provided herein is an enzyme-facilitated process for the extraction of PMPs such as, for example, enzymes, from transgenic plants whereby biomaterial from the transgenic plant is collected and treated with a hydrolytic enzyme to hydrolyze cell wall material. The cell wall hydrolysis takes place at temperatures from 20 C-90 C and pH from 3-9 using enzymes such as cellulases, hemicellulases, pectinases, lipases, lignanases, xylanases, galactomanases, and/or remanases, alone or in combination. More specifically, the hydrolytic enzyme is cell wall- or plant tissue-degrading enzyme.

Unless specifically defined otherwise in this specification, all technical and scientific terms used herein have the same meanings as commonly understood by one of ordinary skill in the art to which this invention belongs. As used herein, the term “endogenous”, when used in reference to a gene, means a gene that is normally present in the genome of cells of a specified organism, and is present in its normal state in the cells (i.e., present in the genome in the state in which it normally is present in nature). The term “exogenous” is used herein to refer to any material that is introduced into a cell. The term “exogenous nucleic acid molecule” or “transgene” refers to any nucleic acid molecule that either is not normally present in a cell genome or is introduced into a cell. Such exogenous nucleic acid molecules generally are recombinant nucleic acid molecules, which are generated using recombinant DNA methods as disclosed herein or otherwise known in the art.

As used herein, the term “nucleic acid molecule” or “polynucleotide” or “nucleotide sequence” refers broadly to a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms are used herein to include naturally occurring nucleic acid molecules, which can be isolated from a cell, as well as synthetic molecules, which can be prepared, for example, by methods of chemical synthesis or by enzymatic methods such as by the polymerase chain reaction (PCR). The term “recombinant” is used herein to refer to a nucleic acid molecule that is manipulated outside of a cell, including two or more linked heterologous nucleotide sequences. The term “heterologous” is used herein to refer to nucleotide sequences that are not normally linked in nature or, if linked, are linked in a different manner than that disclosed. For example, reference to a transgene comprising a coding sequence operatively linked to a heterologous promoter means that the promoter is one that does not normally direct expression of the nucleotide sequence in a specified cell in nature.

In certain aspect, a method provided herein includes mechanical disruption of the plant biomaterial in combination with enzymatic treatment either simultaneously or sequentially. Mechanical disruption devices could utilize grinders, wet mills, hammer mills, burr mills, or any other method for mechanically disrupting plant material. Reducing cell walls using the methods provided herein will facilitate PMPs leaving cells at a more rapid rate. The methods can be combined with an exudate process (see, e.g., U.S. Pat. No. 6,096,546). It will be understand that factors such as temperature, holding time, reaction vessel holding capacity, dose, and pH, can be varied to find optimal conditions for extracting the PMP.

PMPs include proteins, peptides, polypeptides, or other organic molecules (including small molecules) that are made by a process and/or pathway that includes a polypeptide or protein. The term “small molecule” includes any chemical or other moiety, other than proteins, peptides, polypeptides, and nucleic acids, that can act to affect biological processes. Small molecules can include any number of therapeutic agents presently known and used, or can be small molecules synthesized in a library of such molecules for the purpose of screening for biological function(s). Small molecules are distinguished from macromolecules by size. The small molecules of this invention usually have molecular weight less than about 5,000 daltons (Da), preferably less than about 2,500 Da, more preferably less than 1,000 Da, most preferably less than about 500 Da. For example, PMPs such as antibiotics can be recovered from plants using methods provided herein. The PMP is typically expressed from, or produced using, a protein expressed in the plant from an exogenous nucleic acid molecule. The exogenous nucleic acid molecule is typically a transgene. For example, the transgene can be an enzyme involved in the synthesis of the PMP.

In certain aspects, the hydrolytic enzyme used to hydrolyze plant cell wall material during recovery of the PMP is expressed from a transgene within the plant. In these aspects, additional enzyme can optionally be added to isolated plant material to assist with breakdown of the cell wall. In certain aspects, the transgenic enzyme is expressed at a much higher rate than the PMP.

Expression of transgenes in plants is known in the art. For example, plant transformation technologies, such as those using Agrobacterium tumefaciens T-DNA-mediated transformation and ballistic introduction, can be used to generate plants that express a PMP and optionally a hydrolytic enzyme from transgenes. For expression of a transgene, such as a transgene that encodes a PMP or a protein involved in synthesis of a PMP, and/or expression of a transgene that encodes a hydrolytic enzyme used in the methods provided herein, the transgene is typically operably linked to a promoter that is active in plant cells. The promoter can be a constitutive promoter (e.g., an ubiquitin promoter), a tissue-specific promoter, such as a reproductive tissue promoter (e.g., an anther specific promoter such as a tapetum specific promoter), an inducible promoter, or a developmental or stage-specific promoter.

In general, the nucleotides comprising an exogenous nucleic acid molecule (transgene) are naturally occurring deoxyribonucleotides, such as adenine, cytosine, guanine or thymine linked to 2′-deoxyribose, or ribonucleotides such as adenine, cytosine, guanine or uracil linked to ribose. However, a nucleic acid molecule or nucleotide sequence also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234, 1994; Jellinek et al., Biochemistry 34:11363-11372, 1995; Pagratis et al., Nature Biotechnol. 15:68-73, 1997, each of which is incorporated herein by reference). Similarly, the covalent bond linking the nucleotides of a nucleotide sequence generally is a phosphodiester bond, but also can be, for example, a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986, 1994; Ecker and Crooke, BioTechnology 13:351360, 1995, each of which is incorporated herein by reference). The incorporation of non-naturally occurring nucleotide analogs or bonds linking the nucleotides or analogs can be particularly useful where the nucleic acid molecule is to be exposed to an environment that can contain a nucleolytic activity, including, for example, a plant tissue culture medium or in a plant cell, since the modified molecules can be less susceptible to degradation.

A nucleotide sequence containing naturally occurring nucleotides and phosphodiester bonds, can be chemically synthesized or can be produced using recombinant DNA methods, using an appropriate polynucleotide as a template. In comparison, a nucleotide sequence containing nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

An exogenous nucleic acid molecule can include operatively linked nucleotide sequences such as a promoter operatively linked to a nucleotide sequence. The term “operatively linked” is used herein to refer to two or more molecules that, when joined together, generate a molecule that shares features characteristic of each of the individual molecules. For example, when used in reference to a promoter (or other regulatory element) and a second nucleotide sequence encoding a gene product, the term “operatively linked” means that the regulatory element is positioned with respect to the second nucleotide sequence such that the regulatory element effects its function with respect to the second nucleotide sequence in substantially the same manner as it does when the regulatory element is present in its natural position in a genome (e.g., a promoter effects transcription of an operatively linked coding sequence). In another example, two operatively linked nucleotide sequences, each of which encodes a polypeptide (e.g., one encodes a PMP and one encodes a hydrolytic enzyme), can be such that the coding sequences are in frame and, therefore, upon transcription and translation, result in production of two polypeptides, which can be two separate polypeptides or a fusion protein.

Where an exogenous nucleic acid molecule includes a promoter operatively linked to a nucleotide sequence encoding an RNA or polypeptide of interest, the exogenous nucleic acid molecule can be referred to as an expressible exogenous nucleic acid molecule (or transgene). The term “expressible” is used herein because, while such a nucleotide sequence can be expressed from the promoter, it need not necessarily actually be expressed at a particular point in time. For example, where a promoter of an expressible transgene is an inducible promoter lacking basal activity, an operatively linked nucleotide sequence encoding an RNA or polypeptide of interest only is expressed following exposure to an appropriate inducing agent.

Transcriptional promoters generally act in a position and orientation dependent manner, and usually are positioned at or within about five nucleotides to about fifty nucleotides 5′ (upstream) of the start site of transcription of a gene in nature. In comparison, enhancers can act in a relatively position or orientation independent manner, and can be positioned several hundred or thousand nucleotides upstream or downstream from a transcription start site, or in an intron within the coding region of a gene, yet still be operatively linked to the coding region so as to enhance transcription. The relative positions and orientations of various regulatory elements in addition to a promoter, including the positioning of a transcribed regulatory sequence such as an internal ribosome entry site, or a translated regulatory element such as a cell compartmentalization domain in an appropriate reading frame, are well known and methods for operatively linking such elements are routine in the art (see, for example, Sambrook et al., “Molecular Cloning: A laboratory manual” (Cold Spring Harbor Laboratory Press 1989); Ausubel et al., “Current Protocols in Molecular Biology” (John Wiley and Sons, Baltimore Md. 1987, and supplements through 1995), each of which is incorporated herein by reference).

Promoters useful for expressing a nucleic acid molecule of interest can be any of a range of naturally-occurring promoters known to be operative in plants or animals, as desired. The promoters useful in the present invention can include constitutive promoters, which generally are active in most or all tissues of a plant; inducible promoters, which generally are inactive or exhibit a low basal level of expression, and can be induced to a relatively high activity upon contact of cells with an appropriate inducing agent; tissue specific (or tissue preferred) promoters, which generally are expressed in only one or a few particular cell types (e.g., plant anther cells); and developmental or stage specific promoters, which are active only during a defined period during the growth or development of a plant. Exemplary promoters include the constitutive 35S cauliflower mosaic virus (CaMV) promoter, the ripening-enhanced tomato polygalacturonase promoter, the E8 promoter, and the fruit specific 2A1 promoter, U2 and U5 snRNA promoters from maize, the promoter of the alcohol dehydrogenase gene, the Z4 promoter from a gene encoding the Z4 22 kD zein protein, the Z10 promoter from a gene encoding a 10 kD zein protein, a Z27 promoter from a gene encoding a 27 kD zein protein, the A20 promoter from the gene encoding a 19 kD zinc protein, the light inducible promoter derived from the pea rbcS gene and the actin promoter from rice (U.S. Pat. No. 5,641,876; WO 00/70067), or the Maize ubiquitin promoter (Christensen et al., 1992) or maize histone promoter (Rasco-Gaunt et al., Plant Cell Reprod. 2003).

Tissue specific or stage specific regulatory elements further include, for example, the AGL8/FRUITFULL regulatory element, which is activated upon floral induction (Hempel et al., Development 124:3845-3853, 1997, which is incorporated herein by reference); root specific regulatory elements such as the regulatory elements from the RCP1 gene and the LRP1 gene (Tsugeki and Fedoroff, Proc. Natl. Acad., USA 96:12941-12946, 1999; Smith and Fedoroff, Plant Cell 7:735-745, 1995, each of which is incorporated herein by reference); flower specific regulatory elements such as the regulatory elements from the LEAFY gene and the APETELA1 gene (Blazquez et al., Development 124:3835-3844, 1997, which is incorporated herein by reference; Hempel et al., supra, 1997); seed specific regulatory elements such as the regulatory element from the oleosin gene (Plant et al., Plant Mol. Biol. 25:193-205, 1994, which is incorporated herein by reference), and dehiscence zone specific regulatory element. Additional tissue specific or stage specific regulatory elements include the Zn13 promoter, which is a pollen specific promoter (Hamilton et al., Plant Mol. Biol. 18:211-218, 1992, which is incorporated herein by reference); the UNUSUAL FLORAL ORGANS (UFO) promoter, which is active in apical shoot meristem; the promoter active in shoot meristems (Atanassova et al., Plant J. 2:291, 1992, which is incorporated herein by reference), the cdc2a promoter and cyc07 promoter (see, for example, Ito et al., Plant Mol. Biol. 24:863, 1994; Martinez et al., Proc. Natl. Acad. Sci., USA 89:7360, 1992; Medford et al., Plant Cell 3:359, 1991; Terada et al., Plant J. 3:241, 1993; Wissenbach et al., Plant J 4:411, 1993, each of which is incorporated herein by reference); the promoter of the APETELA3 gene, which is active in floral meristems (Jack et al., Cell 76:703, 1994, which is incorporated herein by reference; Hempel et al., supra, 1997); a promoter of an agamous-like (AGL) family member, for example, AGL8, which is active in shoot meristem upon the transition to flowering (Hempel et al., supra, 1997); floral abscission zone promoters; L1-specific promoters; and the like. Additional tissue-specific promoters can be isolated using well-known methods (see, e.g., U.S. Pat. No. 5,589,379).

Inducible promoters demonstrate increased transcriptional activity upon contact with an inducing agent. Inducing agents can be chemical, biological or physical agents or environmental conditions that effects transcription from an inducible regulatory element. In response to exposure to an inducing agent, transcription from the inducible regulatory element generally is initiated de novo or is increased above a basal or constitutive level of expression. An inducing agent useful for inducing expression from an inducible promoter is selected based on the particular inducible regulatory element. Examples of inducible regulatory elements include a metallothionein regulatory element, a copper inducible regulatory element, or a tetracycline inducible regulatory element, the transcription from which can be effected in response to divalent metal ions, copper or tetracycline, respectively (Furst et al., Cell 55:705-717, 1988; Mett et al., Proc. Natl. Acad. Sci., USA 90:4567-4571, 1993; Gatz et al., Plant J. 2:397-404, 1992; Roder et al., Mol. Gen. Genet. 243:32-38, 1994, each of which is incorporated herein by reference). Inducible regulatory elements also include an ecdysone regulatory element or a glucocorticoid regulatory element, the transcription from which can be effected in response to ecdysone or other steroid (Christopherson et al., Proc. Natl. Acad. Sci., USA 89:6314-6318, 1992; Schena et al., Proc. Natl. Acad. Sci., USA 88:10421-10425, 1991, each of which is incorporated herein by reference); a cold responsive regulatory element or a heat shock regulatory element, the transcription of which can be effected in response to exposure to cold or heat, respectively (Takahashi et al., Plant Physiol. 99:383-390, 1992, which is incorporated herein by reference).

Additional regulatory elements useful in the methods or compositions of the invention include, for example, the spinach nitrite reductase gene regulatory element (Back et al., Plant Mol. Biol. 17:9, 1991, which is incorporated herein by reference); a light inducible regulatory element (Feinbaum et al., Mol. Gen. Genet. 226:449, 1991; Lam and Chua, Science 248:471, 1990, each of which is incorporated herein by reference), a plant hormone inducible regulatory element (Yamaguchi-Shinozaki et al., Plant Mol. Biol. 15:905, 1990; Kares et al., Plant Mol. Biol. 15:225, 1990, each of which is incorporated herein by reference), and the like. An inducible regulatory element also can be a plant stress-regulated regulatory element, the copper responsive promoter from the ACEI system (Mett et al., Proc. Natl. Acad. Sci., USA 90:4567-4571, 1993, which is incorporated herein by reference); the promoter of the maize In2 gene, which responds to benzenesulfonamide herbicide safeners (Hershey et al., Mol. Gen. Gene. 227:229-237, 1991; Gatz et al., Mol. Gen. Genet. 243:32-38, 1994, each of which is incorporated herein by reference), and the Tet repressor of transposon Tn10 (Gatz et al., Mol. Gen. Genet. 227:229-237, 1991, which is incorporated herein by reference). Other promoters active in plant cells include a gamma zein promoter, an oleosin ole16 promoter, a globulin I promoter, an actin I promoter, an actin cl promoter, a sucrose synthetase promoter, an INOPS promoter, an EXM5 promoter, a globulin2 promoter, a b-32, ADPG-pyrophosphorylase promoter, an LtpI promoter, an Ltp2 promoter, an oleosin ole 17 promoter, an oleosin ole18 promoter, an actin 2 promoter, a pollen-specific protein promoter, a pollen-specific pectate lyase gene promoter or PG47 gene promoter, an anther specific RTS2 gene promoter, SGB6 gene promoter, or G9 gene promoter, a tapetum specific RAB24 gene promoter, a anthranilate synthase alpha subunit promoter, an alpha zein promoter, an anthranilate synthase beta subunit promoter, a dihydrodipicolinate synthase promoter, a Thi 1 promoter, an alcohol dehydrogenase promoter, a cab binding protein promoter, an H3C4 promoter, a RUBISCO SS starch branching enzyme promoter, an actin3 promoter, an actin7 promoter, a regulatory protein GF14-12 promoter, a ribosomal protein L9 promoter, a cellulose biosynthetic enzyme promoter, an S-adenosyl-L-homocysteine hydrolase promoter, a superoxide dismutase promoter, a C-kinase receptor promoter, a phosphoglycerate mutase promoter, a root-specific RCc3 mRNA promoter, a glucose-6 phosphate isomerase promoter, a pyrophosphate-fructose 6-phosphate-1-phosphotransferase promoter, an ubiquitin promoter, a beta-ketoacyl-ACP synthase promoter, a 33 kDa photosystem 11 promoter, an oxygen evolving protein promoter, a 69 kDa vacuolar ATPase subunit promoter, a glyceraldehyde-3-phosphate dehydrogenase promoter, an ABA-and ripening-inducible-like protein promoter, a phenylalanine ammonia lyase promoter, an adenosine triphosphatase S-adenosyl-L-homocysteine hydrolase promoter, a chalcone synthase promoter, a zein promoter, a globulin-1 promoter, an auxin-binding protein promoter, a UDP glucose flavonoid glycosyl-transferase gene promoter, an NTI promoter, an actin promoter, and an opaque 2 promoter.

In certain aspects, the present invention provides a recovery process for PMPs involving an initial bulk separation of the crop material containing the PMP from the diluent plant materials followed by an enzyme mediated liquefaction and/sacarrification of the separated plant material in order to facilitate enrichment and typically purification of the PMP. The enzymes employed for the hydrolysis of non-desired plant materials can be selected from such classes of enzymes such as amylases, lipases, cellulases, hemicellulases, pectinases and specific proteases.

Provided herein is a transgenic plant that includes both a transgene encoding a PMP or a protein involved in the production of PMPs and a transgene encoding a hydrolytic enzyme used in the present methods. Also provided herein is a seed produced by a transgenic plant provided herein.

In certain embodiments, provided herein is an enzyme-facilitated process for the extraction of PMPs from corn where the PMP is produced within the starch granule. The starch granule is separated from the oil and germ fractions employing industry standard wet milling techniques. The starch granule containing the PMP is then treated with a hydrolytic enzyme, for example a mixture of amylases or other enzymes, in order to liquefy the starch in the granule to low molecular weight glucose oligomers. The resultant mixture is then separated into low and high molecular weight fractions using size-based membranes. The PMP is then isolated from the high or low molecular weight fraction, depending on the molecular weight of the PMP.

In another aspect, provided herein is an enzyme facilitated process for the extraction of PMPs from corn where the PMP is produced within the endosperm fraction, the embryo fraction or the aleuronic layer. The endosperm fraction, embryo fraction, and/or aleuronic layer, are then contacted with a hydrolytic enzyme under conditions to hydrolyze plant cell wall material. The PMP is then isolated.

In another embodiment, provided herein is an enzyme facilitated process for the extraction of PMPs from corn where the initial steeping step is replaced by a bio-steeping process. Typically, the initial step in the wet milling process involves a soaking process known as steeping which loosens the forces holding the separate components together. Steeping is done in a low pH process with high concentrations of lactic acid produced by lactobacillus in a sulfur dioxide environment. This environment will result in degradation of the majority of PMPs released into such an environment. Classes of enzymes such as cellulases and reductases are known to replace steeping. The selection of the appropriate mix of enzyme will effect a release of the target PMP directly into the bio-steeping liquid, for example through a release of the starch granule containing the PMP. The starch granule is then treated with a hydrolytic enzyme, such as a mixture of amylases, in order to liquefy the starch in the granule to low molecular weight glucose oligomers.

All patents and patent applications, publications, scientific articles, and other referenced materials mentioned in this specification are indicative of the levels of skill of those skilled in the art to which the invention pertains, and each of which is hereby incorporated by reference to the same extent as if each reference had been incorporated by reference in its entirety individually. The inventors reserve the right to physically incorporate into this specification any and all materials and information from any such patents and patent applications, publications, scientific articles, electronically available information, and other referenced materials or documents.

The specific methods, plants, and other products and processes described in this specification are representative of preferred embodiments and are exemplary and not intended as limitations on the scope of the invention, and it is understood that the terminology used herein is for the purpose of describing particular embodiments only and is not intended to limit the scope of the present invention which will be limited only by the appended claims. Other objects, aspects, and embodiments will occur to those skilled in the art upon consideration of this specification and are encompassed within the spirit of the invention as defined by the scope of the claims. It is understood that this invention is not limited to the particular materials and methods described, and it will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Also, the terms “comprising”, “including”, “containing”, etc. are to be read expansively and without limitation. It must be noted that as used herein and in the appended claims, the singular forms “a”, “an”, and “the” include plural reference unless the context clearly dictates otherwise.

The terms and expressions that have been employed are used as terms of description and not of limitation, and there is no intent in the use of such terms and expressions to exclude any now-existing or later-developed equivalent of the features shown and described or portions thereof, but it is recognized that various modifications are possible within the scope of the invention as claimed. Thus, it will be understood that although the present invention has been specifically disclosed by preferred embodiments and optional features, modification and/or variation of the disclosed elements may be resorted to by those skilled in the art, and that such modifications and variations are within the scope of the invention as claimed.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method to isolate a plant-made product, comprising:
producing the plant-made product in a transgenic plant, wherein the transgenic plant expresses an exogenous nucleic acid molecule to produce the plant-made product;
contacting tissue of the plant with an enzyme under conditions effective to hydrolyze the cell wall of cells of the plant tissue; and
collecting, and thus isolating, the plant-made product.

2. A method according to claim 1, wherein the plant-made product is selected from the group consisting of a protein and a peptide encoded by the exogenous nucleic acid molecule.

3. A method according to claim 1, wherein the plant-made product is a small molecule whose synthesis in the transgenic plant requires the expression of the exogenous nucleic acid molecule.

4. A method according to claim 1, wherein both the enzyme and the plant-made product are produced in the transgenic plant as a result of expression of exogenous nucleic acid molecules.

5. A method according to claim 1, wherein the enzyme is provided at a concentration of between 0.1% and 10%.

6. A method according to claim 1 further comprising substantially purifying the isolated plant-made product.

7. A transgenic plant, wherein the transgenic plant includes an exogenous nucleic acid molecule encoding an enzyme capable of degrading a plant cell wall component.

8. A transgenic plant according to claim 7, wherein the transgenic plant further comprises a second exogenous nucleic acid molecule whose expression results in production of a plant-made product in at least one tissue of the transgenic plant.

9. A transgenic plant according to claim 8, wherein the plant-made product is selected from the group consisting of a protein and a peptide.

10. A transgenic plant according to claim 8, wherein the plant-made product is a small molecule whose synthesis in the transgenic plant requires the expression of the second exogenous nucleic acid molecule.

11. A method for enhancing recovery of a plant-made product, comprising:
producing the plant-made product in a transgenic plant, wherein at least one tissue of the transgenic plant expresses an exogenous nucleic acid molecule that encodes an enzyme that, under conditions effective to hydrolyze plant cell walls, hydrolyses cell walls of at least a portion of the cells of tissue(s) in which the enzyme is expressed;
placing the transgenic plant, or at least some of the tissue(s) of the transgenic plant in which the enzyme is expressed, under conditions effective to hydrolyze plant cell walls so as to allow the enzyme to hydrolyze the plant cell walls of the tissue at least in part and thereby enhance recovery of the plant-made product; and
collecting the plant-made product.

12. A method according to claim 11 further comprising substantially purifying the isolated plant-made product.

13. A method according to claim 11, wherein the plant-made product is produced in the transgenic plant as a result of expression of an exogenous nucleic acid molecule.

14. A method according to claim 13, wherein the plant-made product is encoded by the exogenous nucleic acid molecule.

15. A method according to claim 14, wherein the plant-made product is selected from the group consisting of a protein and a peptide.

16. A method according to claim 13, wherein the plant-made product is a small molecule whose synthesis in the transgenic plant requires the expression of the exogenous nucleic acid molecule.
